(19) United States
(12) Patent Application Publication
(10) Pub. No.: US 2026/0124218 A1
(43) Pub. Date: May 7, 2026

(54) Comprehensive Cannabinoid Compositions and Methods for the Management of Anxiety Disorders

(71) Applicants: Dante Picazo, Houston, TX (US); Nancy Paola Duarte Delgado, Bogotà (CO); Nancy Rodrigues Das Fontes, Tondela (PT); Stephanie Dona Hartmann, Caracas (VE); Ana Maria Maldonado Vacca, Bogotà (CO); Maria Angelica Clavijo Romero, Santa Marta (CO); Rosangel del Valle Andrades Fuentes, Caracas (VE)

(72) Inventors: Dante Picazo, Houston, TX (US); Nancy Paola Duarte Delgado, Bogotà (CO); Nancy Rodrigues Das Fontes, Tondela (PT); Stephanie Dona Hartmann, Caracas (VE); Ana Maria Maldonado Vacca, Bogotà (CO); Maria Angelica Clavijo Romero, Santa Marta (CO); Rosangel del Valle Andrades Fuentes, Caracas (VE)

(21) Appl. No.: 18/934,360

(22) Filed: Nov. 1, 2024

(51) Int. Cl. A61K 31/00; A61K 9/00; A61K 9/107; A61K 31/593; A61K 36/185; A61K 36/81; A61K 47/10; A61K 47/14; A61K 47/22; A61K 47/24; A61K 47/26

(52) U.S. Cl. CPC A61K 31/658; A61K 9/006; A61K 9/1075; A61K 31/593; A61K 36/3482; A61K 36/81; A61K 47/10; A61K 47/14; A61K 47/22; A61K 47/24; A61K 47/26

(57) ABSTRACT

This disclosure introduces an advanced cannabinoid-based nanoplatform formulation for oral drop administration, designed for fast-acting anxiety control via the sublingual route to maximize bioavailability. The formulation incorporates broad-spectrum CBD, including cannabinoids such as CBD, CBG, and CBC, alongside terpenes, flavonoids, selected herbal extracts, and essential vitamins to enhance therapeutic efficacy. This formulation targets multiple therapeutic endpoints relevant to a range of anxiety disorders, including Panic Disorder, Specific Phobias, Social Anxiety Disorder, Generalized Anxiety Disorder (GAD), and others. Symptoms addressed span from restlessness, nervousness, and fatigue to concentration difficulties, irritability, muscle tension, sleep disturbances, palpitations, excessive sweating, tremors, nausea, and gastrointestinal discomfort. The active compounds act centrally by modulating 5HT1-A serotonin receptors, dopaminergic pathways, and CB1 and CB2 cannabinoid receptors, aiming to reduce neuroinflammation and rebalance neurotransmitters. Together, cannabinoids, herbal extracts, and vitamins synergistically reduce oxidative stress, fostering mental well-being and delivering a comprehensive approach to anxiety management.

### FIELD OF THE DISCLOSURE

The technical field of the disclosure relates to medicinal, therapeutic, and pharmaceutical uses of cannabinoids for managing anxiety disorders. This disclosure introduces therapeutic compositions featuring broad-spectrum CBD enhanced by a blend of cannabinoids, including cannabigerol (CBG) and cannabichromene (CBC), a plant-based herbal extract and essential vitamins designed to support neurotransmitter balance and reduce neuroinflammation. Key ingredients include Ashwagandha root extract and vitamin D to enhance stress management and promote mental well-being. This composition targets GABAergic and serotoninergic neurotransmitter systems while providing a natural, comprehensive strategy for anxiety management. Through the synergistic effects of these bioactive compounds, the formulation aims to support mood regulation, reduce reliance on traditional anxiolytics, and improve quality of life by offering a balanced, plant-based approach to anxiety relief.

### BACKGROUND INFORMATION

The American Psychiatric Association (APA) defines anxiety disorders as conditions marked by excessive fear and anxiety with accompanying behavioral disturbances. Fear typically responds to immediate threats, triggering autonomic defenses and flight behaviors, whereas anxiety anticipates future threats, manifesting muscle tension, vigilance, and avoidant behaviors. Unlike normal, developmentally appropriate fear or transient anxiety, anxiety disorders are excessive and persistent, lasting at least six months in adults and often less in children. These disorders usually develop in childhood and tend to persist without treatment and require a clinician's evaluation of whether the fear or anxiety is disproportionate, considering cultural factors.

Recent studies underscore the significant roles of the amygdala and hippocampus in the pathophysiology of anxiety disorders. The amygdala is central to processing fear-related stimuli, and its hyperactivity is associated with increased vigilance and emotional dysregulation. Meanwhile, the hippocampus is crucial in contextualizing emotional memories and modulating stress responses via its interaction with the hypothalamic-pituitary-adrenal (HPA) axis (van de Poll et al., 2023). Additionally, the bed nucleus of the stria terminalis (BNST), a part of extended amygdala, is essential for sustained threat responses. Understanding the connectivity between these structures, including the medial prefrontal cortex, is vital for deciphering emotional regulation and anxiety experiences (Duval et al., 2015). Disruptions in these neural circuits may contribute to various anxiety disorders, indicating the potential for targeted therapies.

Anxiety appears to result from dysregulation in key neurotransmitter systems coupled with increased sympathetic nervous system arousal. Imbalances in the serotonergic and noradrenergic systems, particularly underactivity in serotonin and overactivity in norepinephrine, are strongly linked to anxiety symptoms, supporting the use of SSRIs and SNRIs as primary treatments (Adwas et al., 2019). Additionally, the GABAergic system remains central to anxiety treatments like benzodiazepines, with recent studies identifying the α2 GABA receptor subtype as a promising target for reducing anxiety with fewer sedative effects. Corticosteroids and cholecystokinin (CCK) also play roles in modulating neural circuits tied to stress and fear processing, suggesting that these systems may be valuable therapeutic targets for anxiety management (Nuss, 2015).

Medications like Selective Serotonin Reuptake Inhibitors (SSRIs), Selective Serotonin-Norepinephrine Reuptake Inhibitors (SNRIs), and benzodiazepines are effective in managing anxiety, but they can also lead to adverse effects. Benzodiazepines, which enhance GABA's calming effects, may cause drowsiness, muscle relaxation, memory issues, and motor skill impairment, with paradoxical effects like irritability in some cases. They may cause tolerance with long-term use, requiring higher doses for the same effect due to receptor adaptations. This tolerance can increase anxiety over time, as symptoms may worsen or persist due to withdrawal-like effects even with ongoing medication. Acute benzodiazepine intoxication is another risk, typically impacting the central nervous system (CNS) with symptoms like decreased consciousness, hypotonia, and hyporeflexia. In severe cases, intoxication can lead to respiratory depression, hypotension, and the risk of broncho-aspiration, particularly when combined with other CNS depressants (Osés, 2003).

Considering the limitations of current anti-anxiety medications, particularly their side effects and the risk of tolerance with long-term use, there is a significant need for alternative approaches that address both the neurochemical and physiological roots of anxiety. Formulations incorporating broad-spectrum CBD are enhanced by phytotherapeutic compounds such as flavonoids and terpenoids, including myrcene, limonene, caryophyllene, linalool, and pinene are emerging as potential solutions. These botanical agents can offer anxiolytic effects by influencing mood regulation, reducing neuroinflammation, and modulating neurotransmitter pathways, while typically presenting fewer adverse effects.

Additionally, incorporating Ashwagandha root extract, known for its regulatory properties, may enhance stress management and support neurotransmitter balance, further complementing the anxiolytic properties of CBD. Vitamin D is also included in this formulation, given its known role in supporting overall brain health and its potential to reduce anxiety symptoms. Together, these elements constitute a comprehensive, plant-based approach to managing anxiety that could reduce reliance on traditional medications by providing a balanced effect on both mind and body, with fewer risks related to tolerance or dependence.

### SUMMARY OF THE DISCLOSURE

This disclosure presents a comprehensive approach to managing symptoms of anxiety through three main embodiments: (1) a cannabinoid-based composition, (2) a method of treatment, and (3) a manufacturing method.

Composition: The primary embodiment is a cannabinoid-based composition comprising a) an active principle that includes Broad-spectrum CBD oil containing a blend of cannabinoids such as cannabidiol (CBD), cannabigerol (CBG), and cannabichromene (CBC); b) an herbal extract of Ashwagandha root; c) essential vitamins like Vitamin D to enhance stress resilience and promote mental well-being; and d) at least two emulsifying agents suitable for oral administration.

This composition addresses anxiety symptoms by supporting neurotransmitter balance, modulating the endocannabinoid system, and reducing neuroinflammation. The synergistic effects of cannabinoids, terpenes, and regulatory target neurotransmitter systems, including GABA and serotonin pathways, promote a calming effect, stabilize mood, and support resilience against stress. Additionally, Ashwagandha and vitamin D contribute to stress management and emotional stability.

Method of Treatment: This embodiment describes a method of treatment for patients with anxiety involving daily administration of the composition in oral drop form. Each dose of oral drops contains Broad-spectrum CBD oil with terpenes that address symptoms of anxiety, including nervousness, tension, and difficulty relaxing. Using Broad-spectrum CBD oil with nanoplatforms to enhance bioavailability ensures optimal neurotransmitter support, mood balance, and reduction of neuroinflammation. The vitamin D and herb extract, including Ashwagandha root, fortify stress resilience, improve overall quality of life, and reduce reliance on traditional anxiolytics.

Manufacturing Method: This disclosure includes a manufacturing process for the composition, involving precise integration of Broad-spectrum CBD oil, regulatory, and essential vitamins with solubilizing agents on nanoplatforms. Techniques like sonication and high homogenization enhance bioavailability, stability, and consistency for effective oral administration. This therapeutic approach offers a safe, plant-based option for managing anxiety.

By addressing key neurochemical pathways, it aims to alleviate anxiety symptoms and support emotional and cognitive health without the adverse effects often associated with conventional anxiolytics. In essence, this disclosure introduces an evidence-based formulation that supports resilience to stress and improves the quality of life for individuals with anxiety.

### DETAILED DESCRIPTION

### Definitions

Anxiety is an emotion that, when it intensifies and becomes persistent, becomes a disorder, which shares characteristics of excessive fear and anxiety, as well as associated behavioral alterations, such as a feeling of restlessness, nervousness, or excessive worry about future situations, real or imaginary, and can manifest itself both physically (tachycardia, sweating, muscle tension) and psychologically (recurrent negative thoughts, difficulty concentrating) (APA, 2014).

Anxiety disorders encompass a range of conditions characterized by the specific objects, situations, or thoughts that trigger fear or anxiety, as well as the behaviors people adopt to manage or avoid these feelings. The most prevalent anxiety disorders include:

Panic Disorder: Characterized by recurrent episodes of sudden, intense fear, often without a clear trigger.

Specific Phobias: Marked by an intense, persistent fear of particular objects or situations.

Social Anxiety Disorder: Involves a significant fear of social situations where individuals worry about embarrassment or judgment.

Generalized Anxiety Disorder (GAD): Defined by excessive and ongoing worry about various aspects of daily life, GAD often coexists with other anxiety or depressive disorders. Symptoms include both physical manifestations (e.g., palpitations, fatigue, tremors, nausea) and psychological signs (e.g., restlessness, difficulty concentrating, excessive worry, and a pervasive sense of fear) (APA, 2014).

As for the treatment of anxiety disorders, it is usually non-pharmacological treatment, psychotherapy, and medication, although, in some cases, psychotherapy is often combined with pharmacological treatment.

There is a system that adjusts the imbalance that occurs in anxiety disorders, regulating homeostatic balance and maintaining the physiological functions of the body. This system is known as the Endocannabinoid System (ECS) and is composed of cannabinoid receptors, endocannabinoids, and enzymes involved in regulating the production and release of endocannabinoids, causing reactions in the cells. When an alteration occurs, the human body synthesizes endocannabinoids that usually interact with cannabinoid receptors. This interaction leads to the activation of molecular pathways within the cells that cause stability in vital and physiological processes (Di Marzo & De Petrocellis, 2006).

To date, the cannabinoid receptors CB1 and CB2 are the best characterized. They differ in how they transmit the signal and their distribution in different tissues. Both are G protein-coupled receptors (GPCRs) and their activation leads to the inhibition of adenylyl cyclase, which prevents the conversion of ATP to cyclic AMP (CAMP) and prevents specific voltage-sensitive calcium channels, in addition to stimulating mitogen-activated protein kinases (MAP kinases) and inwardly rectifying potassium channels (GIRKs), and recruiting beta-arrestins, among other actions (Howlett & Abood, 2017).

At the molecular level, cannabinoids exert therapeutic effects by activating CB1 and CB2 receptors. CB1 receptors are found mainly in brain, spinal cord, and peripheral nervous system neurons. However, they are also present in specific peripheral organs and tissues, such as endocrine glands, salivary glands, leukocytes, spleen, and heart, and in some regions of the reproductive, urinary, and gastrointestinal tracts. CB1 receptors in the brain modulate neurotransmitters such as serotonin and dopamine, leading to an anxiolytic effect

CB2 receptors, located mainly in immune cells, are also found to a limited extent in the central nervous system. When activated, they inhibit the release of the neurotransmitters glutamate and GABA, which contribute to reducing inflammation. Therefore, pharmacological manipulation of rCB2 could be considered of therapeutic interest in the management of anxiety, reducing the anxiogenic response (Garcia, 2011).

Endogenous and exogenous cannabinoids act on the cannabinoid receptor type 1 (CB1), the serotonin receptor type 1A (5HT1A), and the transient receptor potential vanilloid type 1 (TRPV1). Cannabinoid receptor type 1 agonists have a biphasic effect: they have anxiolytic properties at low doses and anxiogenic properties at high doses. Activation of the CB1 receptor produces an inhibitory effect on the neuron, leading to an anxiolytic effect (Blessing, E. M. 2015).

CBD is a potentially effective treatment for panic disorder, generalized anxiety disorder, and social anxiety disorder, as a single dose administration attenuated and reduced the response of anxiety symptoms to a public speaking test in subjects with social anxiety disorder (Blessing, E. M. 2015).

Oral administration of CBD has been shown to attenuate the anxiogenic effect. CBD also has anxiolytic properties associated with an action on the limbic and paralimbic brain areas (Fusar-Poli et al., 2009).

Broad-spectrum CBD products contain various cannabinoids from the Cannabis plant such as Cannabigerol (CBG), a non-psychotomimetic cannabinoid derived from Cannabis sativa, which shows potential for managing anxiety through several mechanisms. CBG acts as a partial agonist at CB1 and CB2 receptors, which play key roles in regulating mood and stress responses and interacts with serotonin (5-HT1A) receptors, which are critical in anxiety modulation. This interaction with serotonin suggests that CBG may help stabilize mood and reduce anxiety symptoms.

Additionally, CBG's anti-inflammatory and antioxidant effects protect neural cells from oxidative stress, a factor often linked to anxiety (Borrelli, 2013). Studies show that CBG can reduce pro-inflammatory cytokines and reactive oxygen species, contributing to neuroinflammation and oxidative stress in the brain (Gugliandolo, 2018). Together, these properties highlight CBG as a promising natural alternative for anxiety management.

Cannabichromene (CBC), one of the primary cannabinoids in Cannabis sativa, demonstrates therapeutic potential for managing anxiety through its interactions within the endocannabinoid system and other receptor pathways. Although CBC shows a low binding affinity for CB1 receptors, it effectively agonizes CB2 receptors, potentially reducing inflammation, a contributor to anxiety disorders (Udoh, 2019). CBC also inhibits endocannabinoid reuptake, preserving natural endocannabinoid levels and possibly stabilizing mood. Additionally, CBC's activation of TRPA1 channels suggests antinociceptive and stress-relieving effects, while preclinical studies highlight its antidepressant and neuroprotective properties, both relevant for anxiety management (De Petrocellis, 2012).

Terpenes are natural compounds with diverse medicinal properties. They are found in both plants and animals and exhibit anxiolytic effects (Gershenzon 2007). Among the most abundant terpenes, we find limonene. Limonene is an organic compound that has been shown to have anxiolytic properties. Research has revealed that limonene increases locomotor activity, suggesting an effect opposite to the immobility associated with anxiety, increases the preference for open spaces in elevated plus maze tests, and mice treated with limonene showed a greater tendency to explore open areas, which is indicative of an anxiety reduction, modulates neurotransmission influencing the production of dopamine, a key neurotransmitter in the regulation of mood and reward and increases the expression of Arc (activity-regulated cytoskeletal-associated protein), which contributes to its anxiolytic effects. (Song, Y. 2021).

Pinene is one of the most abundant terpenes in nature. The anxiolytic effects of α-pinene and β-pinene may occur through mechanisms involving 5-HT1A, β-adrenergic and D1 receptors, as well as increased synthesis of hippocampal BDNF (Brain-derived Neurotrophic Factor) and midbrain dopamine; systems that have been identified as key players in the pathogenesis of anxiety (Weston-Green et al., 2021).

Myrcene (β-Myrcene) is the most prevalent terpene in modern Cannabis chemotypes. It is characterized by having therapeutic properties for managing anxiety since it can enhance GABA activity, reducing anxiety. It also has a narcotic effect mediated by α-2 adrenoreceptors (Chen, 2024), promoting a state of relaxation and reducing anxiety.

Caryophyllene is a natural compound present in many plants. It has been shown to have anxiolytic properties by activating CB2 receptors, triggering mechanisms that lead to calming and relaxing effects.

Linalool is an organic compound that has been shown to have the potential to relieve anxiety. It can interact with the GABAergic system, enhancing the effect of the neurotransmitter, promoting relaxation, and reducing anxiety. It also modulates the release of neurotransmitters such as serotonin, dopamine, and norepinephrine, helping to restore neurochemical balance (Harada, H. 2018).

Flavonoids are phytochemical compounds in many plants, fruits, vegetables, and leaves, and they have potential medical applications. Flavonoids possess several medicinal benefits, including anticancer, antioxidant, anti-inflammatory, and antiviral properties (Ullah et al., 2020). They also have neuroprotective effects and exert actions on multiple molecular targets and exhibit anxiolytic action by affecting multiple neuronal transmissions or pathways such as noradrenergic, serotonergic, GABAergic, and dopaminergic; inhibition of monoamine oxidase and tropomyosin B receptor kinase; and simultaneously increase nerve growth and brain-derived neurotrophic factors (Pannu et al., 2021).

The “entourage effect” describes the synergistic interaction between cannabinoids, like CBD, and other compounds in Cannabis, particularly terpenes, and flavonoids. Combined, these compounds enhance each other's therapeutic effects, potentially offering a more comprehensive benefit than isolated cannabinoids alone. Terpenes, such as myrcene, limonene, caryophyllene, linalool, and pinene, may influence how cannabinoids interact with the body's endocannabinoid system, modulating effects like relaxation, anti-inflammation, and mood improvement. This synergy suggests that broad-spectrum Cannabis extracts, which preserve a range of cannabinoids and terpenes, may provide enhanced therapeutic effects compared to isolated compounds.

Ashwagandha, a medicinal herb traditionally used in Ayurvedic medicine, is recognized for its regulatory properties, meaning it helps the body adapt to stress and maintain balance in response to various physical or mental stressors (Speers et al., 2021). The meta-analysis of randomized controlled trials on Ashwagandha supplementation revealed that it significantly reduces anxiety and stress levels compared to placebo. Standardized measures of anxiety and stress were consistently lower in participants taking Ashwagandha, underscoring its potential as a natural intervention for managing these conditions. Higher doses of Ashwagandha also correlated with more substantial benefits, suggesting a dose-response relationship that supports its role in promoting emotional stability and resilience against stress (Akhgarjand et al., 2022).

Vitamin D plays a significant role in mental health, with research indicating that low levels of this nutrient are associated with an increased risk of anxiety and depression. Supplementation may help alleviate symptoms, particularly in deficient individuals (Akpιnar & Karadağ, 2022). It has been found that individuals with low levels of vitamin D experienced notable reductions in anxiety when supplemented, indicating its potential role as a beneficial intervention for anxiety management. These findings suggest that addressing vitamin D deficiency may help enhance emotional well-being and provide a natural strategy for alleviating anxiety, particularly in those deficient in this essential nutrient (Zhu et al., 2020).

### Pharmaceutical Composition:

Aspects of the disclosure provide a methodology for addressing the main symptoms associated with anxiety, aiming towards obtaining the optimal health balance (physical-mental-social) in patients using a nano platform composition of phytocannabinoids (broad spectrum CBD) and related compounds thereof, specifically targeting patients who have a diagnosis of generalized anxiety disorder.

The details outlined in the mentioned examples are broader than those shown since modifications can be made without departing from the scope of the disclosure and the equivalent of the claims. The following description of specific embodiments will help clarify the construction, operation, and additional benefits of the disclosure.

The disclosure pertains to a composition designed for anxiety treatment, which includes a) an active principle comprising at least one cannabinoid; b) a second active principle comprising an herbal extract; c) a vitamin; and d) emulsifying agents. In some embodiments, the active ingredient features at least one cannabinoid selected from various cannabinoids, provided in a broad-spectrum CBD form. It includes cannabidiol (CBD), cannabigerol (CBG), cannabinol (CBN), cannabichromene (CBC), cannabidivarin (CBDV), tetrahydrocannabivarin (THCV), tetrahydrocannabinol acid (THCA), cannabidiol acid (CBDA), cannabigerol acid (CBGA), and cannabichromene acid (CBCA).

These cannabinoids can be used individually or in specific combinations to achieve specific therapeutic effects tailored to the patient's needs, such as the selection of cannabinoids such as CBD.

CBD reduces the phosphorylation of tau protein in PC12 cells, providing a different neuroprotective mechanism during anxiety episodes. Functional magnetic resonance imaging (fMRI) has detected attenuated responses in the amygdala and cingulate cortex induced by CBD (200 mg) while viewing fearful facial stimuli. Furthermore, CBD has been shown to disrupt frontal intrinsic connectivity between the amygdala and anterior cingulate during the neural response to fearful faces. Together, these studies demonstrate the action of CBD in limbic and paralimbic brain areas, which are known to be associated with anxiety.

CBD holds promise in alleviating various symptoms associated with anxiety, including excessive worry, marked by constant restlessness or concern over different situations. It may help reduce muscle tension, which often presents as tightness or stiffness, and address persistent fatigue that lingers despite rest. CBD's effects also improve focus, alleviate difficulty concentrating, and ease irritability and frustration. Additionally, CBD has been shown to support healthier sleep patterns, addressing insomnia and interrupted sleep while mitigating physical symptoms of anxiety, such as palpitations, sweating, tremors, dizziness, or digestive upset. Finally, CBD's potential to calm anxiety could reduce the tendency to avoid situations that trigger anxious feelings.

The broad-spectrum CBD includes multiple minor phytocannabinoids, which can be optimized to leverage the entourage effect, a synergistic interaction that enhances and maximizes each cannabinoid's therapeutic properties. This interaction can significantly amplify effectiveness in reducing inflammation and oxidative stress, ultimately supporting anxiety relief. Broad-spectrum CBD offers unique advantages, providing access to potentially beneficial compounds like terpenes. These aromatic compounds, naturally found in Cannabis and other plants, possess antioxidant, antimicrobial, and anti-inflammatory properties. This formula, containing plant-derived terpenes such as limonene, pinene, caryophyllene, and linalool, has specific benefits that improve the overall effectiveness of the blend.

In another embodiment, Ashwagandha, known as Withania somnifera, is a popular herbal extract in Ayurvedic medicine. Its main components include alkaloids, such as withanine and withasomnine, that have anti-inflammatory and antioxidant properties and saponins that contribute to its regulatory effects and may help reduce stress. Furthermore, it provides fatty acids that support general wellness and phenolic compounds with antioxidant properties. Ashwagandha is primarily used to help reduce stress, improve energy, and boost focus and overall well-being.

Moreover, the nano-platform delivery system ensures that these cannabinoids, terpenes, herbal extracts, and vitamins are delivered efficiently, with enhanced bioavailability and controlled release. This allows the active ingredients to provide sustained relief from anxiety-related symptoms. By tailoring the cannabinoid composition to specific therapeutic targets and utilizing the nano-platform for enhanced delivery, the formulation offers a comprehensive solution to managing the symptoms and progression of anxiety and other related conditions.

In various embodiments, the composition can be adjusted in cannabinoid concentration to fine-tune therapeutic potency and optimize patient response. For demonstration, we present examples at concentrations of 5%, 10% and 20% offering a flexible range for dose escalation, tapering, or specific adjustments based on individual patient needs. This approach allows for precise dosing modifications in response to the treated condition, patient tolerance, and desired therapeutic outcomes. For instance, higher cannabinoid concentrations may be ideal for patients undergoing chronic treatment, while lower concentrations may better suit those requiring acute, shorter-term management.

Furthermore, the ratio of the different cannabinoids within the composition can be adjusted to optimize the therapeutic profile while maintaining efficacy. This customizable approach allows for specific formulation strategies, such as emphasizing the therapeutic effects of certain cannabinoids to target particular symptoms or physiological pathways.

Alternatively, a composition with a balanced ratio of CBD, CBG, and CBC in the broad-spectrum CBD could be designed to address multiple symptoms simultaneously. Broad-spectrum CBD adds another layer of therapeutic potential due to its well-documented interaction with neurotransmitter systems related to anxiety, promoting beneficial brain changes and showing potential to treat several psychiatric conditions, including anxiety. The inclusion of CBD broad spectrum could enhance the overall anti-inflammatory response, reduce anxiety associated with stress responses and overlapping symptoms, and improve sleep quality, which is often impaired in patients with anxiety. Additionally, the composition could include minor cannabinoids such as CBC and CBG to provide a broad-spectrum anti-inflammatory response, which is critical for counteracting oxidative stress.

By carefully adjusting cannabinoid concentrations and ratios, the composition can be tailored to alternative treatments for anxiety and mood disorders while improving overall health and well-being without the side effects commonly associated with traditional anxiolytic medications. Nanoplatform technology facilitates controlled release, maintaining therapeutic levels of cannabinoids in conjunction with terpenes, herbal extracts, and vitamins for an extended period, which is particularly beneficial for conditions such as anxiety. In conclusion, varying the concentration and ratios of cannabinoids and other compounds in the composition allows for a flexible and personalized treatment strategy. This approach maximizes therapeutic benefits through synergistic effects while minimizing potential side effects.

In various embodiments, the composition can vary in cannabinoid concentration to tailor therapeutic potency and patient response, allowing for fine-tuning based on the treated condition, patient tolerance, and desired therapeutic outcome. For example, broad-spectrum CBD contains a rich blend of cannabinoids, including cannabidiol (CBD), cannabigerol (CBG), and cannabichromene (CBC), along with beneficial terpenes-all without the Cannabis plant's psychoactive compound, tetrahydrocannabinol (THC). This combination helps reduce inflammation and oxidative stress linked to anxiety disorders.

The composition can also be adjusted to include CBG, particularly for patients who may benefit from its neuroprotective and antioxidant properties. The addition of CBG may be advantageous because it exhibits among its potential pharmacological targets transient receptor potential (TRP) channels, cyclooxygenase enzymes (COX-1 and COX-2), and cannabinoid receptors, 5-HT1A: a key receptor for the control of anxiety disorders.

Customizing cannabinoid ratios offers a more precise approach to treating various conditions. For example, prioritizing a higher amount of CBC over CBG can enhance activity at the 5-HT1A receptor. This adaptability in formulations allows for individualized treatments that optimize efficacy and reduce adverse effects. In more severe anxiety situations, it is crucial to consider higher concentrations of CBC and CBG to ensure adequate treatment and prevent complications associated with anxiety.

This approach improves symptom relief, reduces inflammation, and supports overall central nervous system health by promoting neuronal balance regeneration and reducing the risk of new anxiety episodes. Using multiple cannabinoids in specific ratios in combination with terpenes, vitamins, and herbal extracts can also help minimize the risk of side effects commonly associated with higher doses of individual cannabinoids, increasing the tolerability of the method of treatment.

In multiple formulations, the composition is crafted with precise concentrations of cannabinoids that are strategically balanced to maximize therapeutic benefits. The cannabinoids in a broad-spectrum CBD are selected for their individual and synergistic properties and their anti-inflammatory and neuroprotective effects.

In one illustrative embodiment, the composition designed for anxiety contains 250 mg/ml of broad-spectrum CBD per dose. However, additional embodiments may include modifications to the formulation, allowing for more precise tailoring to patient needs, therapeutic goals, and tolerability, thereby optimizing treatment efficacy and minimizing potential adverse effects.

A slight decrease in concentration may be beneficial for patients who respond well to cannabinoids but experience minor side effects at higher doses. This approach allows for effective symptom management while reducing the cannabinoid load, facilitating more sustainable tolerance to long-term treatment, which may range from 3 to 6 months. On the other hand, an increase in concentration may be helpful for those who require more effective control of symptoms, significantly if their condition has progressed beyond what the standard dose can relieve, without the need to opt for higher concentrations.

Increased concentration is targeted in patients dealing with chronic anxiety symptoms such as excessive worry, muscle tension, fatigue, difficulty concentrating, irritability, sleep disturbances, physical symptoms, and avoidance of situations. Higher levels of broad-spectrum CBD contribute to enhanced anti-inflammatory and anxiolytic effects.

In other embodiments, the cannabinoid ratio can be adjusted within these concentration ranges to tailor the composition to specific patient needs or therapeutic goals, providing flexibility to manage anxiety symptoms. In another embodiment, the composition can be combined with other phytotherapeutics, such as terpenes, herbal extracts, and vitamins, which have demonstrated effectiveness in addressing anxiety-associated conditions.

In further embodiments, cannabinoid ratios can be modified within these concentration ranges to meet specific patient needs or therapeutic goals, providing a versatile approach to managing various anxiety symptoms. This customizable framework allows the formulation to address various degrees and expressions of anxiety.

In another embodiment, the composition can be combined with key components such as terpenes (anti-inflammatory and anxiolytic effects), Ashwagandha extract (anti-inflammatory and antioxidant properties, helps reduce stress, and promotes overall health), and vitamin D (an essential cofactor in cellular functioning and widely studied in anxiety disorders). These components are crucial to providing comprehensive neuronal support and addressing the pathophysiological pathways of anxiety disorders.

The composition may also include terpenes such as limonene, pinene, caryophyllene, and linalool in certain embodiments. Anxiety affects the central nervous system (CNS) and other systems, including the gastrointestinal tract, tissues, joints, and muscles, often leading to increased inflammation. The combination of cannabinoids and terpenes contents in broad-spectrum CBD, enhances the formulation's anti-inflammatory potential, addressing both anxiety and its related inflammatory responses. Each terpene within broad-spectrum CBD contributes unique therapeutic effects; for instance, early studies on limonene indicate its anxiolytic properties in animal models, attributed to its serotonergic action in the prefrontal cortex and dopaminergic function in the hippocampus, with mechanisms involving 5-HT1A receptors.

Several rodent studies have investigated the efficacy of α-pinene and β-pinene in alleviating anxiety and depressive behaviors, consistently showing positive results. Linalool, another terpene, also shows potential anxiolytic effects within the central nervous system (CNS). In recent research, linalool inhalation produced a significant anxiolytic-like effect in standard behavioral tests on mice, with the absence of this effect in anosmic mice suggesting its mechanism is mediated through olfactory pathways. β-caryophyllene (BCP), a natural sesquiterpene, acts as a selective agonist of CB2 receptors. In a recent study, the anxiolytic activity of BCP was assessed along with its possible mechanisms of action, specifically examining the roles of GABAA/BZD and 5-HT1A receptors.

Pre-treatment with flumazenil, a GABAA receptor antagonist, and NAN-190, a selective 5-HT1A receptor antagonist, was used. The anxiolytic effects of linalool were blocked by flumazenil, indicating its interaction with GABAergic transmission. These findings suggest that specific terpenes (contents in broad spectrum CBD), interact with various neural pathways to produce anxiolytic effects.

By adjusting the ratios of cannabinoids and incorporating herbal extracts and vitamins, this formula offers a comprehensive therapeutic approach for individuals with anxiety. It addresses the complex pathophysiology of the condition, providing symptom relief and sustained support for the central nervous system. This adaptable approach allows for treatment that is finely tuned to each patient's unique needs, delivering an effective, personalized solution for various forms of anxiety.

When designing therapeutic formulas for complex conditions such as anxiety, it is vital to include a wide range of bioactive components that work together synergistically. The ability to modify these substances in various concentrations helps to maximize their therapeutic efficacy, providing a treatment approach that is highly customizable and tailored to each patient's individual needs.

The flexibility to adjust these components in various concentration ranges allows for customized formulations that meet specific patient needs. For example, cannabinoids are in concentrations ranging from 5% to 20% in the broad-spectrum CBD oil. Terpenes (contained in the broad spectrum CBD) play a crucial role in treating anxiety by improving CNS health and influencing the hypothalamus-amygdala, contributing to antidepressant effects, primarily through compounds such as limonene.

Similarly, herbal extracts such as Ashwagandha can be incorporated in concentrations from 45% to 52%. Ashwagandha offers significant benefits for the treatment of anxiety due to its anti-inflammatory and antioxidant properties, effectively addressing anxiety symptoms.

Ashwagandha is a well-known herb extract recognized for its significant benefits in managing anxiety. Its therapeutic effects stem from potent anti-inflammatory and antioxidant properties, which help address physiological imbalances linked to anxiety. When included in formulations at concentrations between 45% and 52%, Ashwagandha plays a key role in stabilizing processes that contribute to anxiety. Its anti-inflammatory properties are particularly valuable, as chronic inflammation has been increasingly associated with anxiety disorders. Research suggests that inflammation may influence neurobiological pathways related to stress and anxiety responses. By reducing inflammation, Ashwagandha can help decrease hyperactivity in these pathways, promoting a calming effect that alleviates anxiety symptoms. Additionally, reducing systemic inflammation supports overall neurological health, which is beneficial for patients whose anxiety symptoms may be resistant to standard treatments.

Oxidative stress results from an imbalance between free radicals and antioxidants in the body, leading to cellular damage that can affect neural function. Ashwagandha's antioxidant compounds neutralize free radicals, protecting neurons from damage and supporting healthy neurotransmitter function. This is essential in the regulation of mood and stress response, as oxidative damage to neural cells has been implicated in both the onset and persistence of anxiety symptoms. By protecting neural integrity, it may support cognitive functions that are often impaired in individuals with anxiety.

The incorporation of Ashwagandha root extract into cannabinoid-based compositions enhances the anxiolytic effects of the formulation through a synergistic approach that addresses the neurochemical and physiological aspects of anxiety. Ashwagandha works alongside cannabinoids by promoting a balanced response to stress and facilitating mood regulation. In summary, the inclusion of this herbal extract plant in substantial concentrations provides a dual-action approach that not only reduces anxiety symptoms but also supports long-term mental well-being by addressing inflammatory and oxidative factors that are often present in anxiety disorders.

In some embodiments, vitamins, specifically vitamin D, are considered crucial for the health of the immune system. A deficiency of vitamin D is a risk factor to cardiovascular disease, cognitive decline, and cancer. Recent research highlights Vitamin D as a crucial cofactor in the treatment of anxiety.

This ability to customize the formulation by adjusting the proportions of these three critical components (broad-spectrum CBD, herbal extracts, and vitamin D) allows for a highly flexible and personalized therapeutic approach.

In a preferred embodiment, the composition contains 25% broad-spectrum CBD. In another embodiment, it includes a 47% concentration of the Ashwagandha root extract. Additionally, in other preferred embodiments, Vitamin D is incorporated at a concentration of 0.01%.

Each of these embodiments provides flexibility in therapeutic design, allowing the composition to be tailored to the severity of the anxiety disorder severity and specific emotional and cognitive symptoms.

Excipients or carriers may be incorporated into a pharmaceutical composition to enhance stability, bioavailability, and patient acceptability, supporting the formulation's efficacy and shelf life. These excipients are selected based on their ability to facilitate the distribution of the active cannabinoids while ensuring the integrity of the mixture. Appropriate excipients can ensure that cannabinoids and other active ingredients in the formulation are effectively delivered to target tissues, maintain stability during storage, and are well tolerated by patients.

In this formulation, excipients may include solubilizers, emulsifiers, stabilizers, antioxidants, lipidic agents, flavoring agents, aqueous systems, and preservatives. For instance, a lipidic agent like Medium-chain triglycerides (MCTs) acts as effective solvents for hydrophobic cannabinoids, such as CBD, ensuring they stay dissolved and readily absorbable upon administration. This enhances bioavailability, as cannabinoids dissolve more efficiently allowing them to remain in a lipophilic solution that optimizes absorption. Solubilizers and emulsifiers, such as Sorbitan 20 and Egg lecithin, promote even distribution of cannabinoids, Ashwagandha, and Vitamin E throughout the formulation, prevent phase separation, and support consistent dosing.

These selected excipients work synergistically to ensure safety, efficacy, and optimal therapeutic outcomes, ultimately enhancing patient adherence. Beyond oral formulations, excipients can be tailored for other administration routes, including parenteral, sublingual, or topical, to meet individual patient needs and therapeutic objectives. For instance, flavoring or sweetening agents can improve the palatability of oral formulations, facilitating patient adherence to therapy. This is particularly important in sublingual formulations designed for immediate therapeutic effects, as they rapidly address anxious symptoms.

In pharmaceutical formulations, especially those administered orally, a considerable part of the composition is made up of excipients. These include preservatives, antioxidants, aqueous agents, and flavoring agents, as well as stabilizers, solubilizers, emulsifiers, and lipid agents, which ensure effective delivery of the active ingredients, maintain long-term stability, and help minimize potential side effects. Typically, these excipients represent a significant percentage of the total formulation, ranging from 27% to 47%.

In formulations where active ingredients represent a minor fraction of the total composition, excipients play a critical role in preserving therapeutic agents' physical and chemical stability. This is especially important in oral formulations, where instability or poor solubility can compromise therapeutic efficacy and increase the risk of adverse reactions. Therefore, maintaining an optimal excipient concentration, often exceeding 27.99%, is essential for effective administration, ensuring consistent bioavailability, and extending the product's shelf life.

In this composition, stabilizing, solubilizing, emulsifying, preservative, and antioxidant agents, both aqueous and lipidic, are present in concentrations ranging from 7.0% to 10%. This high concentration guarantees a homogeneous distribution of the active ingredients, optimizes bioavailability, and ensures the safety and effectiveness of the formulation for oral use.

In different formulations, these excipients may incorporate a variety of functional agents intended to improve stability, efficacy, and overall patient experience. This may include solubilizers, stabilizers, emulsifiers, antioxidants, and preservatives, all collaborating to optimize the delivery of active ingredients such as cannabinoids. Solubilizing agents are essential in formulations containing lipophilic compounds, ensuring that these ingredients are dissolved evenly and consequently improving their bioavailability.

In formulations involving cannabinoids, which are generally lipid-soluble, emulsifying agents play a crucial role when combined with water-based substances. These emulsifiers facilitate the creation of stable mixtures between the oil and water phases, ensuring that the cannabinoids remain evenly dispersed in the formulation. This prevents phase separation and ensures that the active ingredients are delivered consistently in each dose.

Medium-chain triglycerides (MCT) function as lipidic agents that act as a solvent for broad-spectrum hydrophobic cannabinoids. This ensures that these compounds remain dissolved and easily accessible for absorption after administration. MCTs are recognized for their capacity to form homogeneous solutions, a critical property in formulations requiring uniform distribution of active compounds, such as broad-spectrum cannabinoids, to achieve consistent dosing and therapeutic efficacy. This characteristic is especially valuable in enhancing solubility and ensuring the sustained performance of cannabinoids within the formulation.

Additionally, MCT contributes to the overall stability of the formulation by minimizing oxidation and degradation of active cannabinoids. By maintaining cannabinoids in a stable solution with appropriate viscosity and rheological properties, MCTs effectively extend product shelf life, ensuring that therapeutic potency remains consistent throughout the product's designated half-life.

MCT are typically incorporated at concentrations between 8% and 12%, with an optimal level of approximately 10%, to provide an ideal solution while supporting formulation stability. A concentration of 10% MCTs is regarded as optimal for maintaining formulation stability, as it ensures cannabinoids remain evenly dispersed. At this concentration, MCTs help achieve a uniform and stable formulation, ensuring that each dose delivers a consistent amount of active ingredients, thereby enhancing therapeutic reliability for patients with every administration.

In other formulations, a solubilizing system may encompass additional agents such as Sorbitan esters, Egg lecithin, Polysorbates, and Poloxamers. These ingredients work together to develop an effective emulsion system that stabilizes cannabinoids and improves the performance and longevity of the formulation. This stability is especially critical in pharmaceutical formulations containing cannabinoids, which are naturally hydrophobic and require well-formulated emulsions to ensure consistent bioavailability and optimal therapeutic efficacy.

This combination of solubilizers ensures that the formulation remains stable under a variety of conditions, such as temperature fluctuations, agitation, or prolonged storage. Oral formulations require a high level of stability to ensure that cannabinoids are delivered effectively and consistently with each dose.

Emulsifying agents, such as Egg lecithin and Sorbitan (e.g., Sorbitan 20), are critical in pharmaceutical formulations, especially those containing cannabinoids, which are typically lipophilic. These emulsifiers enable the formation of stable and homogeneous liquid mixtures, ensuring that the active ingredients are evenly distributed throughout the solution. This is crucial in oral dosage forms, where consistent and precise delivery of cannabinoids is required to achieve the expected therapeutic effect. Without effective emulsifiers, there is a risk that cannabinoids will separate from the aqueous phase, which can lead to inconsistent dosing and reduce the efficacy of the treatment. Egg lecithin, derived from cholesterol esters, can encapsulate hydrophobic cannabinoids and keep them stably dispersed in aqueous formulations. Sorbitans, such as Sorbitan 20, is a synthetic emulsifier that stabilizes oil-water mixtures, ensuring homogeneity and preventing phase separation.

Egg lecithin and Sorbitan 20 significantly enhance cannabinoid dispersion, promoting efficient oral absorption, particularly in drop formulations. This high bioavailability and rapid metabolic processing make it a valuable solubilizing agent, facilitating cannabinoid transport across cellular membranes for faster absorption and prompt therapeutic onset.

Overall, the inclusion of Egg lecithin and Sorbitan as emulsifying agents at concentrations ranging from 7.0% to 9.0%, with a preferred concentration of 7.0%. In some embodiments, the Egg lecithin is in a range of 4.0% to 7.0% with a preferred concentration of 6.0%. In some embodiments, the Sorbitan 20 is in a range of 1.0% to 3.0% with a preferred concentration of 1.0%. These improve the efficacy, safety, and reliability of cannabinoid-based oral formulations. By improving solubility, preventing separation, and ensuring consistent absorption, Egg lecithin and Sorbitan help optimize the therapeutic delivery of cannabinoids.

In addition, antioxidants aid in cannabinoid-based formulas as these compounds are susceptible to oxidative degradation when exposed to environmental conditions such as light, heat, air, and humidity. Cannabinoids like CBD possess unsaturated bonds within their molecular structure, rendering them susceptible to oxidative degradation. Oxidative stress initiates structural changes within the cannabinoids, often producing inactive or less active metabolites, thereby reducing the overall effectiveness of the formulation.

Antioxidants play a crucial role in preserving the stability of cannabinoids and protecting oxidation-sensitive excipients within the formulation. Lipid-based excipients, for instance, are prone to oxidative degradation upon exposure to oxygen, which can compromise the structural integrity and stability of the entire formulation. The inclusion of antioxidants mitigates this risk by stabilizing these vulnerable components, thereby upholding the product's overall quality and integrity. In certain formulation embodiments, a combination of antioxidants may be employed to establish a more comprehensive protective system against oxidative stress, enhancing the longevity and efficacy of the product.

Excessive use of certain antioxidants may cause instability in the formulation or lead to undesirable interactions with other components. In contrast, insufficient amounts of antioxidants may not provide adequate protection against oxidative degradation of cannabinoids. Therefore, it is critical to achieve an optimal balance that ensures the stability and sustained efficacy of the product.

Tocopherols, known as vitamin E, are key antioxidants in preserving the integrity of cannabinoid formulations, as they neutralize reactive oxygen species (ROS) and free radicals responsible for oxidative degradation. Their lipid-soluble nature allows them to protect the lipid components of the formulation, including the cannabinoids. Tocopherols can intercept and stabilize free radicals within the lipid phase, preventing chain reactions that could lead to the degradation of the entire formulation. By preventing oxidation, tocopherols extend the shelf life of cannabinoid formulations, ensuring that the active ingredients retain their potency and therapeutic efficacy.

In other embodiments, antioxidants may include but are not limited to, vitamin E, ascorbic acid, butylated hydroxyanisole, or combinations thereof. These antioxidants are selected to stabilize the formulation by protecting the cannabinoids from oxidative degradation, thereby preserving potency and extending shelf life. In certain embodiments, vitamin E is included at a concentration ranging from 0.05% to 0.10%, with a preferred concentration of 0.05%. Vitamin E acts as a protective antioxidant to prevent the degradation of cannabinoids and enhances their absorption and bioavailability by supporting the integrity of lipid-based carriers within the formulation. The incorporation of antioxidants such as vitamin E and vitamin C serves to maintain the therapeutic efficacy of cannabinoids over time.

Preservatives, which include propylparaben, methylparaben, benzyl alcohol, and potassium sorbate, are crucial for extending the shelf life of pharmaceutical formulations by preventing microbial contamination. This is particularly relevant in oral, semi-solid, and liquid forms, which have a higher risk of bacterial, fungal, and mold growth due to their moisture content.

In other embodiments, preservatives may include, but are not limited to propylparaben, methylparaben, or combinations thereof. These preservatives stabilize the formulation by protecting it from degradation, thereby preserving potency and extending shelf life. In certain embodiments, Propylparaben, and Methylparaben are included at a concentration ranging from 0.1% to 0.3%. In one preferred embodiment, the concentration for these preservatives is 0.20%. In some embodiments, the methylparaben is preferred at 0.18%. In some embodiments, the propylparaben is preferred at 0.02%.

This combination of preservatives and antioxidant agents is essential to maintain the stability of formulations designed for long-term storage or regular use. Preservatives protect against microbial contamination, and antioxidant agents help maintain the chemical integrity of the active ingredients. This is particularly critical in liquid and semi-solid formulations, where the likelihood of degradation is increased due to moisture and other reactive substances. By utilizing these excipients, the formulation can maintain safety, stability, and efficacy throughout its shelf life, providing patients with a reliable treatment alternative.

In various embodiments, the composition may incorporate an aqueous phase vehicle, essential for balancing the formulation and enhancing the solubility of active ingredients consisting of Glycerin and Purified Water. This vehicle is included at concentrations between 10% and 30%, with an optimal concentration of 10.44%. It provides a medium for dissolving or dispersing other components and optimizing the viscosity and fluidity. This water-based vehicle maintains the necessary consistency for the intended application, supporting efficient absorption and distribution of active ingredients.

Likewise, an aqueous phase vehicle contributes significantly to patient comfort and tolerability. Pharmaceutical water serves as a biocompatible carrier that reflects the hydrophilic nature of the human body.

Beyond contributing to stability and solubility, the aqueous phase vehicle increases the solubility of various components, and thus, the overall efficacy of the mixture.

The use of an oil phase vehicle, ranging from 8.0% to 12%, is critical to the success of the formulation. It consists of Medium Chain Triglycerides and its preferred concentration is 10%. This component not only improves the solubility and bioavailability of the cannabinoids, but also ensures the stability and uniformity of the mixture and guarantees a comfortable experience for the patient at the time of administration. By acting as a neutral and biocompatible medium, the oil phase vehicle promotes the administration of consistent and reliable doses, which enhances the efficacy and safety of cannabinoid-based treatments, particularly in oral formulations.

In some embodiments, the composition includes cannabinoids (Broad spectrum CBD), Ashwagandha root extract, and vitamin D as active ingredients in a concentration of 72.01%. In another embodiment, the composition comprises cannabinoids ranging from 5.0% to 25%, Ashwagandha extract ranging from 45% to 50%, and vitamin D ranging from 0.01% to 0.02% of oral solution. In a preferred embodiment, the composition comprises cannabinoids such as Broad spectrum CBD, herbal plants such as Ashwagandha extract, and vitamins such as Vitamin D. In a further preferred embodiment, the composition comprises Broad spectrum CBD at 25%, Ashwagandha root extract at 47%, and Vitamin D at 0.01%.

In another embodiment, the composition additionally contains emulsifying agents. In a further embodiment, the emulsifier agents range from 7.0% to 9%. In a preferred embodiment, the composition comprises emulsifier agents such as Egg lecithin and Sorbitan 20. In a further preferred embodiment, the composition comprises 6.0% egg lecithin and sorbitan at 1.0%.

This pharmaceutical composition has been designed to offer multiple routes of administration and is presented in various pharmaceutical forms, providing a versatile framework for the delivery of cannabinoids and other activities. This approach not only optimizes therapeutic efficacy but also adapts to patients' individual needs and preferences. The possibility of customizing the formulation according to different routes of administration enriches its applicability to treat various medical conditions, facilitating adjustments in dosage, onset of action, and patient comfort.

In another variant, the routes of administration may include oral, sublingual, topical, inhaled, parenteral, and unique topical methods, each offering particular benefits depending on the pathology in question and the therapeutic effects required. Oral administration, through options such as tablets, capsules, or syrups, allows for a systemic distribution of the active ingredients, resulting in a more gradual onset but longer-lasting effects. In contrast, the sublingual route provides a faster effect, bypassing the digestive tract and allowing the ingredients to be absorbed directly into the bloodstream through the mucous membranes. Topical formulations, such as creams and gels, are ideal for localized treatment of conditions, offering direct relief without widespread exposure to the active ingredients.

Inhalation administration via aerosols or sprays allows for rapid absorption of cannabinoids into the respiratory system, making it an ideal choice for conditions that demand immediate relief, such as acute anxiety. In contrast, parenteral routes, which comprise subdermal, subcutaneous, intravenous, intramuscular, and intra-articular injections, offer precise and rapid delivery of the active ingredients. This method is especially effective for conditions that require rapid intervention, such as anxiety and other psychiatric disorders.

This composition shows great versatility in its pharmaceutical forms, allowing the creation of tablets, capsules, oral drops, syrups, suspensions, powders for reconstitution, elixirs, creams, emulsions, oral solutions, gummies, candies, sprays, oral topicals, mouthwashes, sublingual tablets, sublingual films, ovules, suppositories, injectable solutions, and injectable suspensions. Each form offers unique advantages related to stability, patient adherence, and the delivery profile of the active ingredients. For example, sublingual films and tablets are a discreet and convenient method that avoids first-pass metabolism, while gummies and candies offer an attractive alternative that encourages compliance, especially among children and the elderly.

The pharmaceutical composition is preferably designed for oral administration and is offered as a soft gel. This approach is particularly effective for delivering cannabinoids, allowing them to exert intense anti-inflammatory and neuronal effects. Oral formulations are often used in the treatment of anxiety and other mood disorders, providing targeted relief from imbalances and reducing side effects at a systemic level.

Bioequivalence also plays an essential role by emphasizing the flexibility and adaptability of this pharmaceutical composition in different forms and routes of administration. This legal principle allows for patent protection that extends beyond specific embodiments, encompassing equivalent variations that perform the same function in essentially the same way and achieve the same result. For example, a composition intended for oral use may have an equivalent formulation for parenteral or topical administration, which is also covered by the patent if it achieves the same therapeutic effect with similar ingredients or methods.

This versatility allows the composition to be tailored to the needs of different patient groups and medical conditions, without losing its essence. For example, a formulation initially created as an oral solution in drops, later adapted for oral use through advanced bioavailability technologies, could be considered an equivalent under this doctrine, providing similar therapeutic effects through a different administration method.

In summary, the design of the pharmaceutical composition provides flexibility in multiple administration routes and a wide range of pharmaceutical forms, demonstrating its versatility for various therapeutic applications. The incorporation of bioequivalence reinforces this adaptability, ensuring that even subtle changes in administration methods are protected.

### Treatment Protocol:

According to one embodiment, the composition can be adapted for various routes of administration beyond oral, depending on the patient's condition and the desired therapeutic outcomes. These routes encompass topical and transdermal preparations, thus offering adaptable treatment strategies. Furthermore, the composition can be formulated in multiple dosage forms, such as solutions, emulsions, or nanoemulsions, to maximize bioavailability and promote patient compliance.

The composition can also be formulated with different controlled release mechanisms, including immediate, sustained, or delayed release, to further enhance therapeutic experience. These options allow for the gradual release of active cannabinoids, such as broad-spectrum CBD, over time, improving efficacy and reducing dosing frequency. This adaptability gives healthcare providers the ability to customize treatment based on the severity of the anxiety disorder, patient feedback, and individual medical considerations.

The compositions described in this disclosure may be presented in numerous pharmaceutical dosage forms, customized to improve patient compliance and optimize therapeutic efficacy in various healthcare settings. These forms include solid, semi-solid, liquid, and gaseous preparations, each precisely designed to target distinct routes of administration, ensuring adaptability in treatment and maximizing therapeutic benefits.

In one embodiment, the pharmaceutical composition containing phytocannabinoids is designed to be compatible with multiple routes of administration, accommodating a variety of therapeutic contexts. These methods may include oral, sublingual, topical, inhalation, parenteral, and intravaginal administration. Each route provides treatment flexibility and precise dose control, enhancing bioavailability and ensuring that the cannabinoids reach their intended sites of action to address diverse patient needs.

In this embodiment, capsules, tablets, and solutions (drops) are highly convenient oral formulations, especially for patients looking for an easy-to-use option for managing chronic conditions over the long term. For faster results, sublingual forms such as solutions (drops), sprays, and soluble films bypass gastrointestinal metabolism, allowing cannabinoids to be absorbed directly into the bloodstream. Both oral and sublingual delivery methods are ideal for systemic treatments, as they offer prolonged bioavailability of the active compounds.

In one embodiment, topical formulations, including transdermal patches, are designed to release the active compounds into the body through the skin. Transdermal patches are particularly effective as they offer a steady and controlled release of cannabinoids as they enter the bloodstream, making them suitable for alleviating anxiety symptoms.

The use of inhalation via aerosols or vaporized cannabinoid products offers a convenient and fast-acting method of delivering therapeutic benefits. This route allows cannabinoids to be rapidly absorbed through the lungs, making it ideal for immediate relief of acute symptoms such as palpitations, shortness of breath, chest pressure or pain, dizziness, fear of losing control, or feelings of impending doom. By bypassing first-pass metabolism, inhalation provides rapid delivery of cannabinoids into the bloodstream with a nearly instantaneous onset of effects.

Parenteral cannabinoid formulations, designed for routes such as intradermal, intra-articular, subcutaneous, intravenous, intramuscular, or intrathecal, allow for precise and controlled delivery, especially in clinical settings where rapid therapeutic effects are needed. Intravenous formulations ensure rapid systemic administration of cannabinoids for immediate relief during acute anxiety attacks, while subcutaneous and intramuscular injections provide a slower, sustained release, offering prolonged therapeutic effects for chronic conditions.

Additionally, the composition can take advantage of nanotechnology-based delivery systems to increase bioavailability and ensure more precise tissue targeting. Nanoparticles, nanoemulsions, or liposomes can help protect cannabinoids from degradation and enhance their absorption across biological barriers, including the blood-brain barrier or the skin. These advanced delivery systems allow for more efficient absorption and retention of cannabinoids at therapeutic doses for extended periods, resulting in more effective relief with minimal side effects.

The compositions of this disclosure may be available in liquid forms and may include syrups, solutions, emulsions, or suspensions. Suitable for oral, sublingual, or topical administration, liquids offer ease of dosing and rapid absorption, making them a suitable option for patients seeking quick relief or who have difficulty swallowing solid forms.

In a further embodiment, the composition could be administered in gaseous forms, such as through inhalers or aerosols, which allows the cannabinoids to be rapidly absorbed through the respiratory tract, providing a fast-acting and efficient method of achieving systemic effects, making it highly suitable for the relief of acute symptoms related to anxiety disorders.

In some configurations, dosage forms may offer immediate-release, sustained-release, controlled-release options, or delayed release allowing for precise control over cannabinoid bioavailability throughout the day. Immediate-release formulations provide rapid symptom relief for acute cases, while sustained-release forms such as nanoparticle-based delivery, release active ingredients gradually over time.

Controlled-release formulations, meanwhile, deliver cannabinoids at a steady rate, optimizing levels in the body for effective chronic symptom control and improved patient adherence. Delayed release formulations can be customized for conditions requiring time-specific or localized treatment. These adaptable release options address a variety of therapeutic needs, from rapid relief to ongoing anxiety symptom management.

### Method of Treatment:

The method of treatment is presented as a solution for sublingual administration. Each drop contains 250 mg/mL of Broad-spectrum CBD oil, Ashwagandha root extract at 470 mg/mL, and Vitamin D at 0.13 mg/mL. The method of treatment is designed for targeted delivery of these bioactive compounds to the application site, offering an option for anxiety.

The treatment method consists of sublingual drops formulated for the management of anxiety disorders. It aims to regulate key neurotransmitters such as serotonin, dopamine, and GABA, thus reducing neuroinflammation and oxidative stress, leading to mood regulation and improved cognitive function.

In this embodiment, these concentrations are designed to provide effective relief of anxiety symptoms such as palpitations, shortness of breath, anxiety and/or phobias with specific or non-specific fears.

In additional embodiments, this formulation exerts its effects via modulation of the endocannabinoid system (ECS), specifically through interaction with cannabinoid receptors CB1 and CB2 expressed within the central nervous system. These receptors play a critical role in mediating symptom evolution and severity in anxiety disorders. The composition comprises active phytocannabinoids, terpenes within a broad-spectrum CBD matrix, as well as essential vitamins and selected herbal extracts, synergistically targeting ECS pathways to enhance therapeutic efficacy.

In one embodiment, the pharmaceutical composition contains compounds such as broad-spectrum CBD (cannabidiol), which includes minor phytocannabinoids such as CBG (cannabigerol), and CBC (cannabichromene).

In some embodiments, these cannabinoids are beneficial for treating symptoms such as palpitations, shortness of breath, anxiety and/or phobias with specific or non-specific fears, through the modulation of neurotransmitters involved in the pathogenesis of anxiety disorders.

In one embodiment, the treatment symptoms for adult patients of any gender, presenting with anxiety-related symptoms, and is intended for individuals aged 18 to 65 years.

In one embodiment, the composition comprising nanoplatforms with phytocannabinoids is presented in oral drops and administered sublingually divided into 2 daily doses, one every 12 hours with a treatment duration of 3 months based on symptom-based monitoring. In one embodiment, the method involves administering an appropriate concentration of the active ingredient of 72.01% per dose. In one embodiment, the method involves administering 6 to 8 drops containing 720.13 mg of the active ingredients per mL every 12 hours for 3 months to 6 months.

In other embodiments, the treatment method may vary based on the dosing regimen and schedule, which are critical to balance efficacy with tolerability. In other embodiments, a single-dose treatment method may be used in which a high concentration of phytocannabinoids or other active ingredients is administered to provide rapid relief of anxiety symptoms.

In other modalities, adaptive treatment plans for anxiety disorders may be designed to adjust the dosage or suggest a combination of therapies in response to changes in the patient's symptoms or overall health. This approach ensures personalized care by modifying the treatment regimen based on improvements or worsening of symptoms, thereby optimizing the efficacy and safety of the therapy throughout the treatment process.

Specificity in Correlation of Dose and Efficacy for Anxiety Management: The current Detailed Description provides essential background on the use of broad-spectrum CBD, Ashwagandha, and Vitamin D for managing anxiety symptoms, detailing concentrations and ratios that define a versatile therapeutic profile.

Expanded Description on Dose-Efficacy Relationships. Defining Effective Concentration Ranges Based on Symptom Severity: For effective treatment, a well-defined range of concentrations can help tailor doses for patients experiencing mild, moderate, or severe anxiety symptoms. For instance, the use of 6.25% to 25% CBD concentration, combined with Ashwagandha at 45% to 50% and Vitamin D at 0.01% to 0.02%, provides a framework for adaptation. Evidence from clinical and preclinical studies suggests that low doses of cannabinoids are often adequate for mild anxiety, while higher doses may be necessary for more persistent or severe cases. To this end, the following examples can illustrate how varying concentrations affect specific anxiety symptoms:

Mild Anxiety: Concentrations at the lower end of the range (e.g., 6.25% CBD and 45% Ashwagandha) may offer sufficient support by interacting with GABA receptors and reducing neuroinflammation, providing subtle mood regulation effects suitable for managing occasional anxiety or general unease.

Moderate to Severe Anxiety: Mid-to-high concentrations (e.g., 12.5% to 20% CBD and 47% Ashwagandha) exhibit enhanced anxiolytic properties, leveraging the synergy between cannabinoids and Ashwagandha's adaptogenic effects to help stabilize mood, modulate neurotransmitters like serotonin, and reduce cortisol levels more effectively. This dosage range aligns with findings indicating that GABAergic activity and endocannabinoid system modulation are more pronounced at these concentrations, supporting robust anxiety relief.

Chronic or Refractory Anxiety: For severe cases, a higher CBD concentration (20% to 25%) and Ashwagandha concentration around 50% can sustain stronger anxiolytic effects by continuously modulating neurochemical pathways associated with chronic anxiety, such as GABA and serotonin. Higher doses of broad-spectrum CBD, which may include minor cannabinoids like CBG and CBC, contribute additional neuroprotective and antioxidant properties, supporting long-term neural stability and resilience to anxiety-related stress.

Dose-Response Curve and Therapeutic Window: Expanding the Detailed Description to include a dose-response curve concept would further support dose recommendations. For broad-spectrum cannabinoids, the anxiolytic response is often biphasic: at low doses, they can reduce anxiety, while at excessively high doses, anxiety may reemerge. Introducing a therapeutic window specific to the broad-spectrum CBD and Ashwagandha composition-such as 6.25% to 25% CBD and 45% to 50% Ashwagandha-would clarify the balance between efficacy and safety, demonstrating that the doses fall within a range proven to maximize therapeutic benefit while minimizing risk of adverse effects or diminished efficacy.

Correlation of CBD and Ashwagandha Synergy on Anxiety-Related Biomarkers: Clinical evidence underscores that CBD, particularly in broad-spectrum form, significantly reduces cortisol and improves heart rate variability, both indicators of anxiety reduction. Ashwagandha's adaptogenic properties complement this by lowering cortisol through modulation of the hypothalamic-pituitary-adrenal (HPA) axis, enhancing resilience to stress. A detailed reference to these biomarkers and the expected dose-related changes would make the connection between dose, bioactivity, and therapeutic effect more explicit.

Consideration of Patient-Specific Factors and Dosage Adjustments: A key element in dosing cannabinoid-based compositions is individual patient variability. Expanding on patient-specific factors (e.g., age, tolerance to cannabinoids, and specific anxiety profiles) and providing a flexible dosage adjustment model would ensure that the composition is applicable to a broader patient population. For instance, younger patients or those with lower tolerance levels might benefit from starting at the lower end of the dosage range, while those with chronic anxiety or higher tolerance could find efficacy in higher concentrations within the therapeutic window.

Practical Dosage Recommendations and Administration Regimen: A structured administration regimen based on these considerations might include starting doses at the lower range (e.g., 6-8 drops containing 6.25% CBD, every 12 hours for two weeks), with incremental increases based on patient feedback and symptom improvement. For patients with persistent symptoms, the regimen could then progress to 12-16 drops of a mid-range concentration (e.g., 12.5%-20% CBD), while monitoring biomarkers such as cortisol and heart rate.

Entourage Effect in Cannabinoid-Based Anxiety Treatments: The entourage effect refers to the hypothesized synergy between cannabinoids, terpenes, and flavonoids, suggesting that these compounds work together more effectively than isolated cannabinoids alone. Broad-spectrum CBD contains not only CBD but also other minor cannabinoids (such as CBG and CBC), terpenes (like limonene and linalool), and flavonoids, all of which have specific therapeutic properties. Scientific research supports that when combined, these compounds act on multiple receptor pathways, achieving a more balanced effect on neurotransmitter modulation and reducing anxiety more effectively than single-compound solutions. This effect aligns with recent studies showing that cannabinoid synergy can result in significant enhancements in mood stabilization and stress reduction at lower dosages, mitigating the risk of adverse side effects common in higher doses of isolated cannabinoids.

Cannabinoid-Cannabinoid Synergy: Cannabinoids like CBG and CBC interact with CBD to enhance anxiolytic responses through modulation of the GABAergic and serotonergic systems. CBG, for instance, acts on serotonin receptors (5-HT1A), a primary target in anxiety management, supporting mood regulation and neuroprotective effects. Meanwhile, CBC indirectly amplifies the effects of CBD by inhibiting endocannabinoid reuptake, thus sustaining increased levels of the body's natural endocannabinoids (e.g., anandamide). This synergy allows the cannabinoids to work in concert, maintaining neurotransmitter balance without requiring high doses of any single cannabinoid, a key feature in reducing potential side effects such as sedation or tolerance.

Terpene-Cannabinoid Synergy: Terpenes like limonene and linalool contribute anxiolytic effects independently but, in combination with cannabinoids, enhance the effectiveness of the formulation by modulating dopaminergic and serotonergic pathways. Limonene has been shown to increase dopamine availability, which improves mood and reduces stress, while linalool acts on GABA receptors, promoting a calming effect. This synergy is significant in cannabinoid-based treatments for anxiety, as it enables the combination to act on multiple neurotransmitter er systems simultaneously, leading to a more comprehensive reduction in anxiety symptoms. By illustrating how each terpene complements the cannabinoid actions, the document would reinforce the unique advantages of the composition over single-compound treatments.

Specific Mechanisms of Action in Anxiety-Related Neurotransmission: CBD primarily targets CB1 receptors in the central nervous system, promoting anxiolytic effects by inhibiting neurotransmitter release related to anxiety. However, when combined with CBG, CBC, and terpenes, this response is enhanced through interactions with receptors like GABA and 5-HT1A, as well as transient receptor potential (TRP) channels associated with stress response modulation. By activating these multiple pathways, the combination is able to achieve a more extensive impact on anxiety symptoms, particularly in stress-related arousal and autonomic response reduction.

Example of Synergistic Mechanisms: In a preclinical model, CBD alone demonstrated mild anxiolytic effects; however, when paired with CBG and limonene, the composition significantly reduced stress-induced elevations in cortisol, suggesting enhanced modulation of the hypothalamic-pituitary-adrenal (HPA) axis. This synergy likely stems from CBG's serotonergic effects, which complement CBD's effects on endocannabinoid signaling, and limonene's dopamine enhancement, collectively stabilizing mood and attenuating the physiological stress response.

Synergy Across Neurochemical Pathways: By acting on dopaminergic, serotonergic, and endocannabinoid systems, the synergy of cannabinoids and terpenes produces an “entourage effect” that affects mood regulation comprehensively, which is especially valuable for treating generalized anxiety disorder (GAD) where multiple neurotransmitters are often dysregulated.

Supporting Evidence and Benefits of a Balanced Cannabinoid-Terpene Ratio: The presence of cannabinoids and terpenes in specific ratios allows the composition to provide therapeutic benefits at doses lower than would be required by single-compound formulations, which in turn reduces the risk of side effects and ensures a safer treatment option for chronic administration. Broad-spectrum CBD, for instance, harnesses this ratio to maximize efficacy without excessive sedation or desensitization, which can occur with higher doses of isolated cannabinoids like THC or CBD.

Entourage Effect's Impact on Biomarker Modulation: the combination of CBD, CBG, and limonene is known to reduce pro-inflammatory cytokines, decrease cortisol, and improve heart rate variability, all of which are measurable indicators of reduced anxiety. By correlating specific biomarker improvements with the entourage effect, the document would offer tangible evidence that the combination approach optimizes patient outcomes.

Tailoring the Composition for Specific Anxiety Subtypes: Anxiety disorders encompass a range of subtypes, each with unique physiological and neurochemical characteristics. The composition's broad-spectrum CBD, Ashwagandha, and Vitamin D blend offers flexibility that could benefit patients with distinct anxiety subtypes, including generalized anxiety disorder (GAD), panic disorder, social anxiety disorder, and specific phobias. By targeting both the neurochemical pathways and physical symptoms associated with each subtype, the composition can deliver specialized relief for the following:

Generalized Anxiety Disorder (GAD): GAD is marked by persistent worry and physiological symptoms like muscle tension and fatigue. The composition's CBD and CBG components directly interact with serotonin and GABA pathways, reducing neurochemical imbalances linked to chronic anxiety. Ashwagandha further complements this by regulating cortisol and HPA axis activity, reducing physical symptoms and promoting emotional stability. Together, these ingredients work to alleviate both the mental and physical aspects of GAD, offering a comprehensive approach to symptom management.

Panic disorder: Panic disorder typically involves acute, episodic symptoms such as palpitations, chest pain, and shortness of breath. The composition's rapid-acting sublingual delivery method provides immediate relief by promoting faster cannabinoid absorption, allowing quick modulation of anxiety-inducing pathways. CBD's anxiolytic effect, combined with limonene's dopamine-enhancing properties, helps stabilize mood and reduces the risk of sudden onset of panic symptoms. This multimodal approach offers effective and immediate relief tailored to the episodic nature of panic attacks.

Social Anxiety Disorder: Social anxiety disorder is driven by heightened vigilance and emotional dysregulation in social situations. This composition addresses the core mechanisms of social anxiety through the combination of CBD and Ashwagandha, which together modulate stress-related neurotransmitters and reduce inflammation. The addition of Vitamin D promotes neural health, further supporting emotional resilience in social environments. The formulation's anxiolytic effects stabilize mood, reduce overactive sympathetic responses, and enhance the patient's ability to manage anxiety-provoking social interactions.

Specific Phobias: Specific phobias, triggered by identifiable situations or objects, often involve a significant physical response. The balanced ratio of CBD and CBG in the composition ensures effective modulation of GABA receptors, promoting a calming effect that mitigates the physical symptoms associated with phobic reactions. Ashwagandha's adaptogenic properties reinforce this effect by supporting the body's physiological response to stress, allowing patients to engage with phobic stimuli with reduced anxiety.

Modulating Key Neurotransmitters and Physiological Responses: To provide effective treatment across various anxiety subtypes, the composition modulates key neurotransmitter systems, primarily GABA, serotonin, and dopamine, which play essential roles in anxiety regulation. The multimodal treatment approach leverages broad-spectrum CBD's versatility in acting on both GABAergic and serotonergic pathways, thereby enhancing its calming and mood-stabilizing effects. Ashwagandha further regulates cortisol levels and the HPA axis, addressing the stress response's physiological component, while terpenes like limonene improve dopamine availability, adding a layer of resilience against depressive symptoms often comorbid with anxiety.

Example of Neurotransmitter Modulation: For patients experiencing chronic, anticipatory anxiety common in GAD and social anxiety, a combination of CBD and Ashwagandha helps maintain balanced serotonin and GABA levels, reducing both anxious thoughts and physical manifestations such as tension and palpitations. The multimodal approach ensures that the formulation addresses not only the symptom's psychological roots but also its physical impact, improving the patient's overall resilience and coping capacity.

Optimizing Bioavailability and Rapid Onset of Action: The nanoemulsion and sublingual delivery format enhances bioavailability, ensuring rapid onset of action. The oil-in-water nanoemulsion promotes efficient absorption, especially beneficial for anxiety subtypes that demand quick relief, such as panic disorder. By offering a variety of dosage strengths within a sublingual format, the composition can accommodate patients who require immediate symptom control as well as those who benefit from sustained release. This flexibility allows healthcare providers to personalize treatments based on the frequency and intensity of symptoms across anxiety subtypes.

Monitoring Patient Responses and Adjusting Treatment: For multimodal treatments, patient monitoring and adaptable dosing schedules are essential to achieve sustained efficacy. By recommending an initial low-dose regimen with gradual dose escalation based on individual response, the composition provides a structure for patient-specific adjustments. Patients presenting with severe, recurring symptoms could begin with higher doses or more frequent sublingual administrations, while those experiencing milder, situational anxiety could utilize lower doses on an as-needed basis. The adaptive nature of this treatment protocol allows patients to gain personalized benefits from the multimodal approach, reducing the likelihood of tolerance and maintaining long-term efficacy.

### Manufacturing Method:

In some embodiments, the fabrication of the formulation necessitates adherence to Good Manufacturing Practices in pharmaceutical products, which refer to liquid products, specifically drops.

In alternative embodiments, the formulation compounds are specifically developed in a liquid drop format. Active ingredients undergo ultrasonic cavitation, creating nanoemulsions designed to enhance bioavailability and therapeutic efficacy.

In another embodiment, nanoemulsions are utilized as a nanoscale emulsion system, offering advantages over traditional emulsions, including enhanced functional properties, increased stability, and improved optical clarity. Ultrasonic or acoustic cavitation generates intense shear forces, supplying the energy required to reduce large droplets to nanometer-sized particles. This process effectively blends multiple liquid phases into a stable, uniform nanometer or submicron emulsion.

In another embodiment, through high-pressure homogenization, intense shear forces are employed to provide the necessary energy to reduce large droplets to nanometer-sized particles, thereby achieving a homogeneous blend of two or more liquid phases in a nanometer or submicron-sized emulsion.

In one embodiment, an emulsion is a biphasic system in which one phase is distributed within the other as minute droplets measuring from 0.1 to 200 nm in diameter. Although this system is thermodynamically unstable, it can be stabilized by the incorporation of an emulsifying agent, commonly known as an emulsifier or emulgent.

In this embodiment, the treatment is prepared as an oil-in-water nanoemulsion, in which the oil phase is dispersed within a continuous water phase. The hydrophobic characteristics of cannabinoids and Vitamin D are harnessed in the oil phase, while the hydrophilic properties of other active components, such as Ashwanganda, are seamlessly incorporated into the water phase.

Nanoparticle technology, in several embodiments, offers a range of benefits for drug delivery, including high drug-loading efficiency, compatibility with combination therapies, controlled release, extended systemic circulation, and enhanced targeted delivery.

### EXAMPLES

### Pharmaceutical Compositions:

The current example discloses a therapeutic agent aimed at treating anxiety. This agent comprises a unique composition that utilizes nanoplatform with broad spectrum CBD.

The example describes a substance designed to treat anxiety in patients. This substance comprises a composition that utilizes nanoplatforms containing cannabinoids, such as CBD. The percentage range for administration is outlined to optimize therapeutic effects.

Particular amounts of cannabinoids: 250 mg/ml of Broad-spectrum CBD oil, containing cannabinoids, such as CBD, CBG, and CBC.

Particular amounts of other active principles: 470 mg/mL of Ashwagandha root extract and 5000 IU (0.13 mg/ml) of Vitamin D.

Excipients: Medium-chain Triglycerides, Alpha Tocopherol (Vitamin E), Methylparaben, Propylparaben, Orange flavor, Glycerin, Sorbitan 20, Egg Lecithin and Purified Water.

Formulation 1: Sublingual Drops 20% CBD (200 mg/mL)

| | Quantity | |
|---|---|---|
| Component | mg/mL | Percentage (%) |
| Broad spectrum CBD (20% CBD) | 250.0 | 25.0% |
| Vitamin D | 0.13 | 0.01% |
| Ashwagandha | 470.0 | 47.0% |
| Medium-chain Triglycerides | 100.0 | 10.0% |
| Alpha Tocopherol (Vitamin E) | 0.50 | 0.05% |
| Methylparaben | 1.80 | 0.18% |
| Propylparaben | 0.20 | 0.02% |
| Orange Flavour | 3.00 | 0.30% |
| Glycerin | 50.0 | 5.0% |
| Sorbitan 20 | 10.0 | 1.0% |
| Egg Lecithin | 60.0 | 6.0% |
| Purified Water | 54.37 | 5.44% |

Formulation 2: Sublingual Drops 10% CBD (100 mg/mL)

| | Quantity | |
|---|---|---|
| Component | mg/mL | Percentage (%) |
| Broad spectrum CBD (10% CBD) | 125.0 | 12.50% |
| Vitamin D | 0.13 | 0.01% |
| Ashwagandha | 470.0 | 47.0% |
| Medium-chain Triglycerides | 120.0 | 12.0% |
| Alpha Tocopherol (Vitamin E) | 0.50 | 0.05% |
| Methylparaben | 1.80 | 0.18% |
| Propylparaben | 0.20 | 0.02% |
| Orange Flavour | 3.00 | 0.30% |
| Glycerin | 70.0 | 7.0% |
| Sorbitan 20 | 30.0 | 3.0% |
| Egg Lecithin | 40.0 | 4.0% |
| Purified Water | 139.37 | 13.94% |

Formulation 3: Sublingual Drops 5% CBD (50 mg/mL):

| | Quantity | |
|---|---|---|
| Component | mg/mL | Percentage (%) |
| Broad spectrum CBD (5% CBD) | 62.5 | 6.25% |
| Vitamin D | 0.13 | 0.01% |
| Ashwagandha | 470.0 | 47.0% |
| Medium-chain Triglycerides | 80.0 | 8.0% |
| Alpha Tocopherol (Vitamin E) | 0.50 | 0.05% |
| Methylparaben | 1.80 | 0.18% |
| Propylparaben | 0.20 | 0.02% |
| Orange Flavour | 3.00 | 0.30% |
| Glycerin | 100.0 | 10.0% |
| Sorbitan 20 | 20.0 | 2.0% |
| Egg Lecithin | 70.0 | 7.0% |
| Purified Water | 191.87 | 19.19% |

### Nanoemulsion Process:

### Example 1: Sublingual Drops at 20% CBD (200 mg/mL)

1. Preparation of the Aqueous phase: In a mixing tank, add 5.44 grams of purified water and heat to approximately 90° C. Add 0.18 grams of Methylparaben and 0.02 grams of Propylparaben to the heated water. Stir until both parabens are fully dissolved in the aqueous phase. Cool between 40° C. to 50° C. Incorporate 5.0 grams of Glycerin, 47.0 grams of Ashwagandha root extract, 0.30 grams of orange flavor, and 6.0 grams of Egg lecithin into the mixture. Stir until all components are fully integrated into the aqueous phase.
2. Preparation of the Oil phase: In a mixing tank, add 10.0 grams of medium-chain triglycerides (MCT) and heat to approximately 40° C. to 50° C. Incorporate 25.0 grams of Broad-spectrum CBD, 0.05 grams of Alpha Tocopherol (Vitamin E), 1.0 gram of Sorbitan 20, and 0.013 grams of Vitamin D. Stir the mixture until all compounds are fully dissolved.
3. Formation of the preliminary emulsion: Slowly add the heated cannabinoid oils to the aqueous phase while stirring vigorously. Use a high-speed homogenizer to ensure proper emulsification of the cannabinoids within the aqueous phase.
4. Formation of the nanoemulsion: To reduce the droplet size in the preliminary emulsion to a range of 50 to 200 nanometers (nm), a high-energy ultrasonic homogenizer is used. The sonication process is carried out in 10-second sonication cycles followed by 10 seconds of rest, for a total period of 15 to 20 minutes. During the entire sonication process, the temperature of the emulsion is maintained at 50° C. Then, cool the nanoemulsion at 30° C.
5. Cooling of the nanoemulsion: After the sonication process is completed, the resulting nanoemulsion is cooled until it reaches a temperature below 30° C., while stirring at a low speed between 30 and 50 rpm, to ensure uniform droplet distribution during cooling.
6. Dispensing into Primary Packaging: Once the nanoemulsion reaches the target temperature, it is dispensed into a 30 mL or 60 mL amber glass primary container suitable for oral administration.

### Example 2: Sublingual Drops at 10% CBD (100 mg/mL)

1. Preparation of the Aqueous phase: In a mixing tank, add 13.94 grams of purified water and heat to approximately 90° C. Add 0.18 grams of Methylparaben and 0.02 grams of Propylparaben to the heated water. Stir until both parabens are fully dissolved in the aqueous phase. Cool between 40° C. to 50° C. Incorporate 7.0 grams of Glycerin, 47.0 grams of Ashwagandha root extract, 0.30 grams of orange flavor, and 4.0 grams of Egg lecithin into the mixture. Stir until all components are fully integrated into the aqueous phase.
2. Preparation of the Oil phase: In a mixing tank, add 12.0 grams of medium-chain triglycerides (MCT) and heat to approximately 40° C. to 50° C. Incorporate 12.50 grams of Broad-spectrum CBD, 0.05 grams of Alpha Tocopherol (Vitamin E), 3.0 grams of Sorbitan 20, and 0.013 grams of Vitamin D. Stir the mixture until all compounds are fully dissolved.
3. Formation of the preliminary emulsion: Slowly add the heated cannabinoid oils to the aqueous phase while stirring vigorously. Use a high-speed homogenizer to ensure proper emulsification of the cannabinoids within the aqueous phase.
4. Formation of the nanoemulsion: To reduce the droplet size in the preliminary emulsion to a range of 50 to 200 nanometers (nm), a high-energy ultrasonic homogenizer is used. The sonication process is carried out in 10-second sonication cycles followed by 10 seconds of rest, for a total period of 15 to 20 minutes. During the entire sonication process, the temperature of the emulsion is maintained at 50° C. Then, cool the nanoemulsion at 30° C.
5. Cooling of the nanoemulsion: After the sonication process is completed, the resulting nanoemulsion is cooled until it reaches a temperature below 30° C., while stirring at a low speed between 30 and 50 rpm, to ensure uniform droplet distribution during cooling.
6. Dispensing into Primary Packaging: Once the nanoemulsion reaches the target temperature, it is dispensed into a 30 mL or 60 mL amber glass primary container suitable for oral administration.

### Example 3: Sublingual Drops at 5% CBD (50 mg/mL)

1. Preparation of the Aqueous phase: In a mixing tank, add 19.19 grams of purified water and heat to approximately 90° C. Add 0.18 grams of Methylparaben and 0.02 grams of Propylparaben to the heated water. Stir until both parabens are fully dissolved in the aqueous phase. Cool between 40° C. to 50° C. Incorporate 10.0 grams of Glycerin, 47.0 grams of Ashwagandha root extract, 0.30 grams of Orange flavor, and 7.0 grams of Egg lecithin into the mixture. Stir until all components are fully integrated into the aqueous phase.
2. Preparation of the Oil phase: In a mixing tank, add 8.0 grams of medium-chain triglycerides (MCT) and heat to approximately 40° C. to 50° C. Incorporate 6.25 grams of Broad-spectrum CBD, 0.05 grams of Alpha Tocopherol (Vitamin E), 2.0 grams of Sorbitan 20, and 0.013 grams of Vitamin D. Stir the mixture until all compounds are fully dissolved.
3. Formation of the preliminary emulsion: Slowly add the heated cannabinoid oils to the aqueous phase while stirring vigorously. Use a high-speed homogenizer to ensure proper emulsification of the cannabinoids within the aqueous phase.
4. Formation of the nanoemulsion: To reduce the droplet size in the preliminary emulsion to a range of 50 to 200 nanometers (nm), a high-energy ultrasonic homogenizer is used. The sonication process is carried out in 10-second sonication cycles followed by 10 seconds of rest, for a total period of 15 to 20 minutes. During the entire sonication process, the temperature of the emulsion is maintained at 50° C. Then, cool the nanoemulsion at 30° C.
5. Cooling of the nanoemulsion: After the sonication process is completed, the resulting nanoemulsion is cooled until it reaches a temperature below 30° C., while stirring at a low speed between 30 and 50 rpm, to ensure uniform droplet distribution during cooling.
6. Dispensing into Primary Packaging: Once the nanoemulsion reaches the target temperature, it is dispensed into a 30 mL or 60 mL amber glass primary container suitable for oral administration.

1. A composition for the treatment of anxiety, comprising:
a) an active principle that includes Broad-spectrum CBD oil comprising a blend of cannabinoids;
b) a second active principle comprising an herbal extract;
c) a third active principle comprising a vitamin;
d) at least two emulsifying agents;
wherein the composition is suitable for oral administration.

2. The composition according to claim 1, wherein the Broad-spectrum CBD oil comprises one or more of the following: cannabidiol (CBD), cannabigerol (CBG), cannabichromene (CBC), terpenes, or combinations thereof.

3. The composition according to claim 2, wherein the Broad-spectrum CBD oil is present in a concentration in the range of 6.25% to 25%.

4. The composition according to claim 1, wherein herbal extracts are in a concentration in the range of 45% to 50%, and vitamins in the range of 0.01% to 0.02%.

5. The composition according to claim 1, wherein the emulsifying agents are Egg Lecithin, Sorbitan, or combinations thereof and are present in a concentration in the range of 7.0% to 9.0%.

6. The composition described in claim 1, wherein the nanoplatform has an average particle size of less than 200 nm and is formulated to ensure the controlled release of cannabinoids and other active ingredients over a sustained period.

7. The composition of claim 1, wherein the formulation is provided as an oral nanoemulsion dosage in drop form.

8. The composition according to claim 1, wherein the herbal extract is Ashwagandha extract, and the vitamin is Vitamin D.

9. A method for treating anxiety in a human individual, comprising: administering an effective concentration of the composition of claim 1, wherein the active principle comprises at least two cannabinoids, at least one herbal extract enriched with Vitamins, and at least two emulsifying agents.

10. The method of claim 9, wherein the human subject is selected for treatment based on the diagnosis of anxiety.

11. The method of claim 10, wherein the individual is selected for treatment of anxiety disorders, including Panic Disorder, Specific Phobias, Social Anxiety Disorder, and Generalized Anxiety Disorder (GAD).

12. The method of claim 9 wherein further comprises identifying individuals using: medical history, physical examination, symptom diary, psychiatric and psychological symptoms, and additional psychiatric tests.

13. The method of claim 1, wherein the individual is identified by having alterations in restlessness or nervousness, difficulty concentrating, irritability, muscle tension, sleep problems, palpitations, excessive sweating, tremors, nausea, or stomach discomfort.

14. The method according to claim 9, wherein the effective concentration is administered as 6 to 8 drops, every 12 hours for 15 days (2 weeks), then increases to 12 to 16 drops daily, every 12 hours for 3 to 6 months.

15. The method according to claim 9, wherein the administration of the effective concentration of the composition acts as a monotherapy for treating anxiety.

16. The method of claim 9, wherein the treatment composition alleviates: restlessness or nervousness, difficulty concentrating, irritability, muscle tension, sleep problems, palpitations, excessive sweating, tremors, nausea, or stomach discomfort.

17. A process for manufacturing the composition for treating anxiety of claim 1, wherein the process comprises:
a. Combining broad-spectrum cannabidiol (CBD) oil which contains additional cannabinoids including cannabigerol (CBG) and cannabichromene (CBC), terpenes, and flavonoids to form an active cannabinoid mixture;
b. Incorporating herbal extract into the active cannabinoid mixture to support the neural axis;
c. Utilizing at least two emulsifying agent to enhance the bioavailability and stability of the composition for oral administration;
d. Subjecting the mixture to ultrasonic cavitation or high-pressure homogenization to create a nanoemulsion, wherein the nanoemulsion provides consistent dispersion of cannabinoids and herbal extract within the composition; and
e. Formulating the resulting nanoemulsion into an oral solution for oral administration, wherein the solution composition is effective in modulating neurotransmitter levels and reducing neuroinflammation associated with anxiety symptoms.

18. A therapeutic composition for treating anxiety of claim 1, comprising:
a. Broad-spectrum CBD in a concentration ranging from 6.25% to 25%;
b. Herbal extract in a concentration ranging from 45% to 50%;
c. Vitamin D in a concentration ranging from 0.01% to 0.02%; and
d. Emulsifying agents in a concentration ranging from 7.0% to 9.0%.

19. A therapeutic composition for treating anxiety, comprising:
a. Broad-spectrum cannabidiol (CBD) at a concentration of 25%;
b. Herbal extract at a concentration of 47%;
c. Vitamin D at a concentration of 0.01%;
d. Egg Lecithin at a concentration of 6.0%; and
e. Sorbitan at a concentration of 1.0%,
wherein the composition is formulated for oral administration.

20. The composition according to claim 2, wherein the terpene are selected from myrcene, limonene, caryophyllene, linalool, pinene or combinations thereof.
